(19) United States
(12) Patent Application Publication
(10) Pub. No.: US 2026/0065557 A1
(43) Pub. Date: Mar. 5, 2026

(54) SYSTEM AND METHOD FOR SUBSPACE PARALLEL IMAGING IN K-SPACE

(71) Applicant: GE Precision Healthcare LLC, Waukesha, WI (US)

(72) Inventor: Robert Marc Lebel, Calgary (CA)

(21) Appl. No.: 18/989,531

(22) Filed: Dec. 20, 2024

Related U.S. Application Data
(60) Provisional application No. 63/689,002, filed on Aug. 30, 2024.

(51) Int. Cl. G06T 11/00

(52) U.S. Cl. CPC G06T 12/10; G06T 12/20; G06T 2210/41; G06T 2211/424; G06T 2211/441

(57) ABSTRACT

A computer-implemented method includes obtaining, via a processing system including one or more processors, multi-channel k-space data of a subject acquired with a magnetic resonance imaging scanner. The computer-implemented method also includes utilizing, via the processing system, subspace convolutional kernels on the multi-channel k-space data to combine information from local neighboring k-space locations and across multiple channels to generate subspace compressed k-space data having fewer channels than a number of channels utilized to acquire the multi-channel k-space data.

### CROSS REFERENCE TO RELATED APPLICATIONS

This application is a Non-Provisional Application claiming priority to U.S. Provisional Patent Application No. 63/689,002, entitled “System And Method For Subspace Parallel Imaging In K-Space”, filed Aug. 30, 2024, which is herein incorporated by reference.

### BACKGROUND

The subject matter disclosed herein relates to medical imaging and, more particularly, to a system and a method for subspace parallel imaging in k-space.

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During magnetic resonance imaging (MRI), when a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, or “longitudinal magnetization”, M_{z}, may be rotated, or “tipped”, into the x-y plane to produce a net transverse magnetic moment, Mₜ. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

Parallel imaging can be in image space (e.g., utilizing sensitivity encoding (SENSE) or eigenvector-based iterative self-consistent parallel imaging reconstruction (ESPIRIT)) or in k-space (e.g., utilizing generalized autocalibrating partial parallel acquisition (GRAPPA), autocalibrating reconstruction for Cartesian imaging (ARC), or SPIRIT). Image space versions of parallel imaging compress multiple channels down to single channel combined image. K-space versions of parallel imaging reconstruct all channels, which are later combined in image space. Channel combination has the benefit of constraining the solution to small subspace. This is particularly beneficial in iterative and regularized implementations. Unfortunately, these methods fail if there is unexpected aliasing in the reconstructed image (i.e., the reconstructed field of view (FOV) is too small). ESPIRIT derives multiple sensitivity maps (i.e., a larger subspace) to handle additional aliasing, but is limited by computational and phase singularities that cause artifacts. K-space versions are more robust to aliasing but operate in multi-channel space, which is computationally intense and poses challenges for effective regularization leveraging of correlated information between channels.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method is provided. The computer-implemented method includes obtaining, via a processing system including one or more processors, multi-channel k-space data of a subject acquired with a magnetic resonance imaging scanner. The computer-implemented method also includes utilizing, via the processing system, subspace convolutional kernels on the multi-channel k-space data to combine information from local neighboring k-space locations and across multiple channels to generate subspace compressed k-space data having fewer channels than a number of channels utilized to acquire the multi-channel k-space data.

In another embodiment, a system is provided. The system includes a memory encoding processor-executable routines. The system also includes a processing system including one or more processors and configured to access the memory and to execute the processor-executable routines, wherein the process-executable routines, when executed by the processing system, cause the processing system to perform actions. The actions include obtaining multi-channel k-space data of a subject acquired with a magnetic resonance imaging scanner. The actions also include utilizing subspace convolutional kernels on the multi-channel k-space data to combine information from local neighboring k-space locations and across multiple channels to generate subspace compressed k-space data having fewer channels than a number of channels utilized to acquire the multi-channel k-space data.

In a further embodiment, a non-transitory computer-readable medium, the computer-readable medium including processor-executable code that when executed by a processing system including one or more processors, causes the processing system to perform actions. The actions include obtaining multi-channel k-space data of a subject acquired with a magnetic resonance imaging scanner. The actions also include utilizing subspace convolutional kernels on the multi-channel k-space data to combine information from local neighboring k-space locations and across multiple channels to generate subspace compressed k-space data having fewer channels than a number of channels utilized to acquire the multi-channel k-space data.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 illustrates a schematic diagram of a magnetic resonance imaging (MRI) system suitable for use with the disclosed techniques;

FIG. 2 is a schematic diagram of an image processing system, in accordance with aspects of the present disclosure;

FIG. 3 is a schematic diagram of an overview of parallel imaging in k-space, in accordance with aspects of the present disclosure;

FIG. 4 is a schematic diagram of SPIRIT;

FIG. 5 is a schematic diagram of subspace parallel imaging in k-space, in accordance with aspects of the present disclosure;

FIG. 6 is a schematic diagram illustrating a process or method for learning or obtaining kernels (e.g., subspace convolutional kernels), in accordance with aspects of the present disclosure;

FIG. 7 is a schematic diagram illustrating a process or method for utilizing kernels (e.g., subspace convolutional kernels), in accordance with aspects of the present disclosure;

FIG. 8 depicts reconstructed multi-channel images, in accordance with aspects of the present disclosure;

FIG. 9 depicts sample k-space and image space before and after reconstruction with subspace parallel imaging (e.g., with fully sampled calibration region), in accordance with aspects of the present disclosure;

FIG. 10 depicts sample k-space and image space before and after reconstruction with subspace parallel imaging (e.g., with undersampled calibration region), in accordance with aspects of the present disclosure; and

FIG. 11 is a flow chart of a method for performing subspace parallel imaging, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion are provided in the context of medical imaging, it should be appreciated that the disclosed techniques are not limited to such medical contexts. Indeed, the provision of examples and explanations in such a medical context is only to facilitate explanation by providing instances of real-world implementations and applications. However, the disclosed techniques may also be utilized in other contexts, such as image reconstruction for non-destructive inspection of manufactured parts or goods (i.e., quality control or quality review applications), and/or the non-invasive inspection of packages, boxes, luggage, and so forth (i.e., security or screening applications). In general, the disclosed techniques may be useful in any imaging or screening context or image processing or photography field where a set or type of acquired data undergoes a reconstruction process to generate an image or volume.

Deep learning (DL) approaches discussed herein may be based on artificial neural networks, and may therefore encompass one or more of deep neural networks, fully connected networks, convolutional neural networks (CNNs), transformer-based networks, unrolled neural networks, perceptrons, encoders-decoders, recurrent networks, wavelet filter banks, u-nets, general adversarial networks (GANs), dense neural networks, or other neural network architectures. The neural networks may include shortcuts, activations, batch-normalization layers, and/or other features. These techniques are referred to herein as DL techniques, though this terminology may also be used specifically in reference to the use of deep neural networks, which is a neural network having a plurality of layers.

As discussed herein, DL techniques (which may also be known as deep machine learning, hierarchical learning, or deep structured learning) are a branch of machine learning techniques that employ mathematical representations of data and artificial neural networks for learning and processing such representations. By way of example, DL approaches may be characterized by their use of one or more algorithms to extract or model high level abstractions of a type of data-of-interest. This may be accomplished using one or more processing layers, with each layer typically corresponding to a different level of abstraction and, therefore potentially employing or utilizing different aspects of the initial data or outputs of a preceding layer (i.e., a hierarchy or cascade of layers) as the target of the processes or algorithms of a given layer. In an image processing or reconstruction context, this may be characterized as different layers corresponding to the different feature levels or resolution in the data. In general, the processing from one representation space to the next-level representation space can be considered as one ‘stage’ of the process. Each stage of the process can be performed by separate neural networks or by different parts of one larger neural network.

The present disclosure provides systems and methods for performing subspace parallel imaging in k-space. The disclosed systems and methods utilize compact k-space parallel imaging kernels that map to and from a compressed subspace. The disclosed systems and methods demonstrate how to obtain and to utilize the compact k-space parallel imaging kernels.

The disclosed embodiments combine the benefit of k-space methods (e.g., robust and dispersed artifacts) with the benefit of image space methods (e.g., constraining the solution to a subspace). The disclosed embodiments provide considerable benefit for iterative model-based methods compared to existing methods.

The disclosed embodiments may allow for reconstruction algorithms that produce better quality images that are robust to aliasing issues. This could allow for higher net acceleration rates than are currently possible. Additionally, the disclosed embodiments may enable faster reconstruction speed since convolutions with compact kernels can be quite fast. In addition, there is no memory overhead of storing large sensitively maps. Further, Fourier transforms are only done in the subspace rather than the full (i.e., all of the channels) multi-channel space, thus, enabling a savings of ten times or more in the Fourier transform time. For the patient, this means faster scans, better images, and more appropriate protocols covering only the anatomy of interest.

The disclosed embodiments include a computer-implemented method that includes obtaining, via a processing system including one or more processors, multi-channel k-space data of a subject acquired with a magnetic resonance imaging scanner. The computer-implemented method also includes utilizing, via the processing system, subspace convolutional kernels on the multi-channel k-space data to combine information from local neighboring k-space locations and across multiple channels to generate subspace compressed k-space data having fewer channels than a number of channels utilized to acquire the multi-channel k-space data (e.g., via convolution or mapping down).

In certain embodiments, the computer-implemented method includes utilizing, via the processing system, complex conjugates of the subspace convolutional kernels to perform transposed convolution on the subspace compressed k-space data to restore the multi-channel k-space data. In certain embodiments, the subspace convolutional kernels include weights that provide consistency when the multi-channel k-space data is mapped down to the subspace compressed k-space data and the subspace compressed k-space data is restored to the multi-channel k-space data. In certain embodiments, the multi-channel k-space as originally acquired provides data consistency with the multi-channel k-space data after restoration.

In certain embodiments, the subspace convolution kernels are learnable. In certain embodiments, the multi-channel space data is undersampled. In certain embodiments, in an iterative manner for a certain number of cycles: performing, via the processing system, data consistency on the multi-channel k-space data after restoration; mapping down, via the processing system, the multi-channel k-space data after restoration to the subspace compressed k-space data utilizing the subspace convolutional kernels; and restoring, via the processing system, the subspace compressed k-space data to the multi-channel k-space data utilizing the complex conjugates of the subspace convolutional kernels; and upon reaching the certain number of cycles, applying, via the processing system, a loss function in one or more locations.

In certain embodiments, the computer-implemented method includes applying the loss function in a multi-channel domain between the multi-channel k-space data as originally acquired and the multi-channel k-space data after restoration. In certain embodiments, the computer-implemented method includes applying the loss function on the subspace compressed data. In certain embodiments, the computer-implemented method includes applying the loss function on the subspace convolutional kernels. In certain embodiments, the computer-implemented method includes updating, via the processing system, parameters or weights of the subspace convolutional kernels utilizing gradient back projection upon reaching the certain number of cycles.

In certain embodiments, the computer-implemented method includes performing, via the processing system, inverse Fourier transformation on the subspace compressed k-space data to generate subspace compressed image data utilizing a smaller number of inverse Fourier transforms than a number of inverse Fourier transforms needed to generate image data from the multi-channel k-space data as originally acquired. In certain embodiments, performing inverse Fourier transformation is part of iterative reconstruction or model-based reconstruction. In certain embodiments, the computer-implemented method includes performing, via the processing system, spatial regularization on the subspace compressed image data. In certain embodiments, the computer-implemented method includes performing, via the processing system, forward Fourier transformation on the subspace compressed image data after spatial regularization to generate the subspace compressed k-space data. In certain embodiments, the computer-implemented method includes performing, via the processing system, forward Fourier transformation on the subspace compressed image data (without prior spatial regularization) to generate the subspace compressed k-space data.

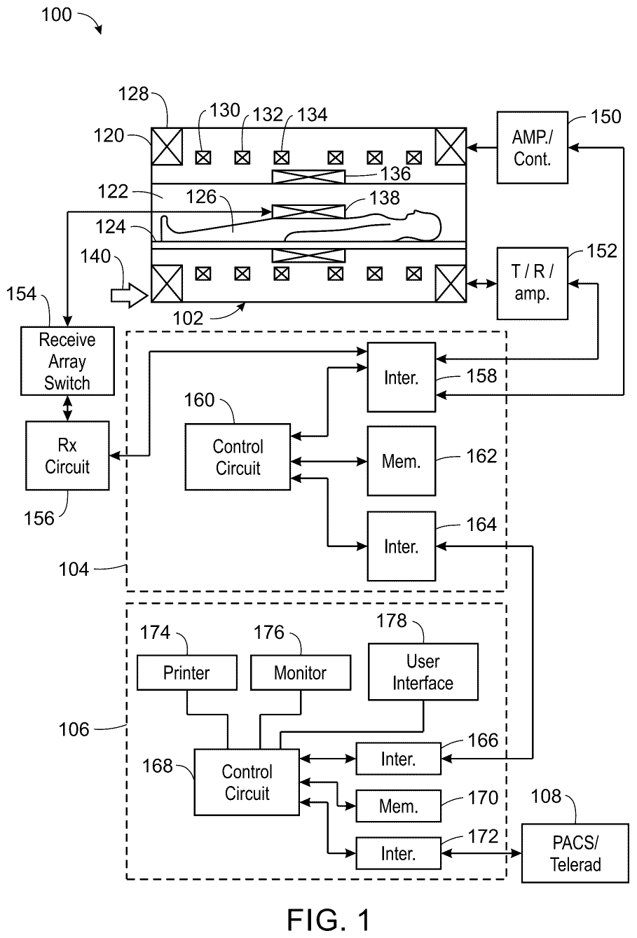

With the preceding in mind, FIG. 1 a magnetic resonance imaging (MRI) system 100 is illustrated schematically as including a scanner 102, scanner control circuitry 104, and system control circuitry 106. According to the embodiments described herein, the MRI system 100 is generally configured to perform MR imaging.

System 100 additionally includes remote access and storage systems or devices such as picture archiving and communication systems (PACS) 108, or other devices such as teleradiology equipment so that data acquired by the system 100 may be accessed on- or off-site. In this way, MR data may be acquired, followed by on- or off-site processing and evaluation. While the MRI system 100 may include any suitable scanner or detector, in the illustrated embodiment, the system 100 includes a full body scanner 102 having a housing 120 through which a bore 122 is formed. A table 124 is moveable into the bore 122 to permit a patient 126 (e.g., subject) to be positioned therein for imaging selected anatomy within the patient.

Scanner 102 includes a series of associated coils for producing controlled magnetic fields for exciting the gyromagnetic material within the anatomy of the patient being imaged. Specifically, a primary magnet coil 128 is provided for generating a primary magnetic field, B₀, which is generally aligned with the bore 122. A series of gradient coils 130, 132, and 134 permit controlled magnetic gradient fields to be generated for positional encoding of certain gyromagnetic nuclei within the patient 126 during examination sequences. A radio frequency (RF) coil 136 (e.g., RF transmit coil) is configured to generate radio frequency pulses for exciting the certain gyromagnetic nuclei within the patient. In addition to the coils that may be local to the scanner 102, the system 100 also includes a set of receiving coils or RF receiving coils 138 (e.g., an array of coils) configured for placement proximal (e.g., against) to the patient 126. As an example, the receiving coils 138 can include cervical/thoracic/lumbar (CTL) coils, head coils, single-sided spine coils, and so forth. Generally, the receiving coils 138 are placed close to or on top of the patient 126 so as to receive the weak RF signals (weak relative to the transmitted pulses generated by the scanner coils) that are generated by certain gyromagnetic nuclei within the patient 126 as they return to their relaxed state.

The various coils of system 100 are controlled by external circuitry to generate the desired field and pulses, and to read emissions from the gyromagnetic material in a controlled manner. In the illustrated embodiment, a main power supply 140 provides power to the primary field coil 128 to generate the primary magnetic field, Bo. A power input (e.g., power from a utility or grid), a power distribution unit (PDU), a power supply (PS), and a driver circuit 150 may together provide power to pulse the gradient field coils 130, 132, and 134. The driver circuit 150 may include amplification and control circuitry for supplying current to the coils as defined by digitized pulse sequences output by the scanner control circuitry 104.

Another control circuit 152 is provided for regulating operation of the RF coil 136. Circuit 152 includes a switching device for alternating between the active and inactive modes of operation, wherein the RF coil 136 transmits and does not transmit signals, respectively. Circuit 152 also includes amplification circuitry configured to generate the RF pulses. Similarly, the receiving coils 138 are connected to switch 154, which is capable of switching the receiving coils 138 between receiving and non-receiving modes. Thus, the receiving coils 138 resonate with the RF signals produced by relaxing gyromagnetic nuclei from within the patient 126 while in the receiving mode, and they do not resonate with RF energy from the transmitting coils (i.e., coil 136) so as to prevent undesirable operation while in the non-receiving mode. Additionally, a receiving circuit 156 is configured to receive the data detected by the receiving coils 138 and may include one or more multiplexing and/or amplification circuits.

It should be noted that while the scanner 102 and the control/amplification circuitry described above are illustrated as being coupled by a single line, many such lines may be present in an actual instantiation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner and either or both of the scanner and system control circuitry 104, 106.

As illustrated, scanner control circuitry 104 includes an interface circuit 158, which outputs signals for driving the gradient field coils and the RF coil and for receiving the data representative of the magnetic resonance signals produced in examination sequences. The interface circuit 158 is coupled to a control and analysis circuit 160. The control and analysis circuit 160 executes the commands for driving the circuit 150 and circuit 152 based on defined protocols selected via system control circuit 106.

Control and analysis circuit 160 also serves to receive the magnetic resonance signals and performs subsequent processing before transmitting the data to system control circuit 106. Scanner control circuit 104 also includes one or more memory circuits 162, which store configuration parameters, pulse sequence descriptions, examination results, and so forth, during operation.

Interface circuit 164 is coupled to the control and analysis circuit 160 for exchanging data between scanner control circuitry 104 and system control circuitry 106. In certain embodiments, the control and analysis circuit 160, while illustrated as a single unit, may include one or more hardware devices. The system control circuit 106 includes an interface circuit 166, which receives data from the scanner control circuitry 104 and transmits data and commands back to the scanner control circuitry 104. The control and analysis circuit 168 may include a CPU in a multi-purpose or application specific computer or workstation. Control and analysis circuit 168 is coupled to a memory circuit 170 to store programming code for operation of the MRI system 100 and to store the processed image data for later reconstruction, display and transmission. The programming code may execute one or more algorithms that, when executed by a processor, are configured to generate a variety of data for training a deep learning-based segmentation model as described below. In certain embodiments, the memory circuit 170 may store one or more neural networks (e.g., deep learning-based reconstruction model such as unrolled deep learning-based reconstruction model). In certain embodiments, the disclosed techniques may occur on a separate computing device having processing circuitry and memory circuitry.

An additional interface circuit 172 may be provided for exchanging image data, configuration parameters, and so forth with external system components such as remote access and storage devices 108. Finally, the system control and analysis circuit 168 may be communicatively coupled to various peripheral devices for facilitating operator interface and for producing hard copies of the reconstructed images. In the illustrated embodiment, these peripherals include a printer 174, a monitor 176, and user interface 178 including devices such as a keyboard, a mouse, a touchscreen (e.g., integrated with the monitor 176), and so forth.

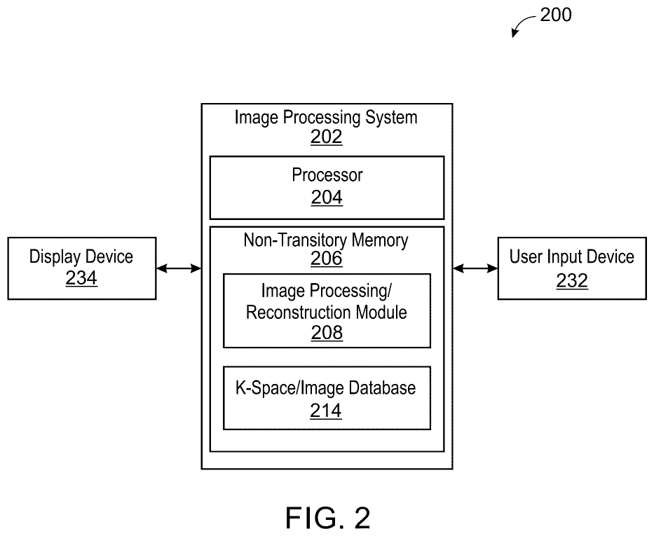

Referring to FIG. 2, an image processing system 202 configured to receive and process k-space data is shown. In some embodiments, the image processing system 202 is incorporated into the MRI system 100. For example, the image processing system 202 may be provided in the MRI system 100 as data processing unit. In some embodiments, at least a portion of image processing system 202 is disposed at a device (e.g., edge device, server, etc.) communicably coupled to the MRI system 100 via wired and/or wireless connections. In some embodiments, at least a portion of image processing system 202 is disposed at a separate device (e.g., a workstation) which can receive k-space data from the MRI system 100 or from a storage device which stores the images/k-space data generated by the MRI system 100. The image processing system 202 may be operably/communicatively coupled to a user input device 232 and a display device 234. User input device 232 may be integrated into an MRI system, such as at user input device of the MRI system 100. Similarly, display device 234 may be integrated into an MRI system, such as at display device of MRI system 100.

The image processing system 202 includes a processor 204 configured to execute machine readable instructions stored in non-transitory memory 206. The processor 204 may be single core or multi-core, and the programs executed thereon may be configured for parallel or distributed processing. In some embodiments, the processor 204 may optionally include individual components that are distributed throughout two or more devices, which may be remotely located and/or configured for coordinated processing. In some embodiments, one or more aspects of processor 204 may be virtualized and executed by remotely-accessible networked computing devices configured in a cloud computing configuration.

Then non-transitory memory 206 may store an image processing/reconstruction module 208 and a k-space/image database 214. The image processing/reconstruction module 208 may obtain the k-space data from the k-space image database. The image processing/reconstruction module 208 is configured to obtain multi-channel k-space data of a subject acquired with a magnetic resonance imaging scanner. The image processing/reconstruction module 208 is configured to utilize subspace convolutional kernels on the multi-channel k-space data to combine information from local neighboring k-space locations and across multiple channels to generate subspace compressed k-space data having fewer channels than a number of channels utilized to acquire the multi-channel k-space data (e.g., via convolution or mapping down).

In certain embodiments, the image processing/reconstruction module 208 is configured to utilize complex conjugates of the subspace convolutional kernels to perform transposed convolution on the subspace compressed k-space data to restore the multi-channel k-space data. In certain embodiments, the subspace convolutional kernels include weights that provide consistency when the multi-channel k-space data is mapped down to the subspace compressed k-space data and the subspace compressed k-space data is restored to the multi-channel k-space data. In certain embodiments, the multi-channel k-space as originally acquired provides data consistency with the multi-channel k-space data after restoration.

In certain embodiments, the subspace convolution kernels are learnable. In certain embodiments, the multi-channel space data is undersampled. In certain embodiments, in an iterative manner for a certain number of cycles: the image processing/reconstruction module 208 is configured to perform data consistency on the multi-channel k-space data after restoration; to map down the multi-channel k-space data after restoration to the subspace compressed k-space data utilizing the subspace convolutional kernels; and to restore the subspace compressed k-space data to the multi-channel k-space data utilizing the complex conjugates of the subspace convolutional kernels; and upon reaching the certain number of cycles, applying, via the processing system, a loss function in one or more locations.

In certain embodiments, the loss function is applied in a multi-channel domain between the multi-channel k-space data as originally acquired and the multi-channel k-space data after restoration. In certain embodiments, the loss function is applied on the subspace compressed data. In certain embodiments, the loss function is applied on the subspace convolutional kernels. In certain embodiments, the image processing/reconstruction module 208 is configured to update parameters or weights of the subspace convolutional kernels utilizing gradient back projection upon reaching the certain number of cycles.

In certain embodiments, the image processing/reconstruction module 208 is configured to perform inverse Fourier transformation on the subspace compressed k-space data to generate subspace compressed image data utilizing a smaller number of inverse Fourier transforms than a number of inverse Fourier transforms needed to generate image data from the multi-channel k-space data as originally acquired. In certain embodiments, the reconstruction comprises iterative reconstruction or model-based reconstruction. In certain embodiments, the image processing/reconstruction module 208 is configured to perform spatial regularization on the subspace compressed image data. In certain embodiments, the image processing/reconstruction module 208 is configured to perform forward Fourier transformation on the subspace compressed image data after spatial regularization to generate the subspace compressed k-space data. In certain embodiments, the image processing/reconstruction module 208 is configured to perform forward Fourier transformation on the subspace compressed image data (without prior spatial regularization) to generate the subspace compressed k-space data.

Non-transitory memory 206 further stores k-space/image database 214. The k-space/image database 214 may include, for example, k-space data acquired via an MRI system and images reconstructed from the k-space data. For example, k-space/image database 214 may store k-space data acquired via MRI system 100, and/or received from other communicatively coupled MRI systems or image databases. In some examples, k-space/image database 214 may store images reconstructed by the image processing/reconstruction module 208.

In some embodiments, non-transitory memory 206 may include components disposed at two or more devices, which may be remotely located and/or configured for coordinated processing. In some embodiments, one or more aspects of non-transitory memory 206 may include remotely-accessible networked storage devices configured in a cloud computing configuration.

User input device 232 may include one or more of a touchscreen, a keyboard, a mouse, a trackpad, a motion sensing camera, or other device configured to enable a user to interact with and manipulate data within image processing system 202. Display device 234 may include one or more display devices utilizing virtually any type of technology. In some embodiments, display device 234 may comprise a computer monitor, and may display MR images. Display device 234 may be combined with processor 204, non-transitory memory 206, and/or user input device 232 in a shared enclosure, or may be peripheral display devices and may comprise a monitor, touchscreen, projector, or other display device known in the art, which may enable a user to view MRI images produced by an MRI system, and/or interact with various data stored in non-transitory memory 206.

It should be understood that image processing system 202 shown in FIG. 2 is for illustration, not for limitation. Another appropriate image processing system may include more, fewer, or different components.

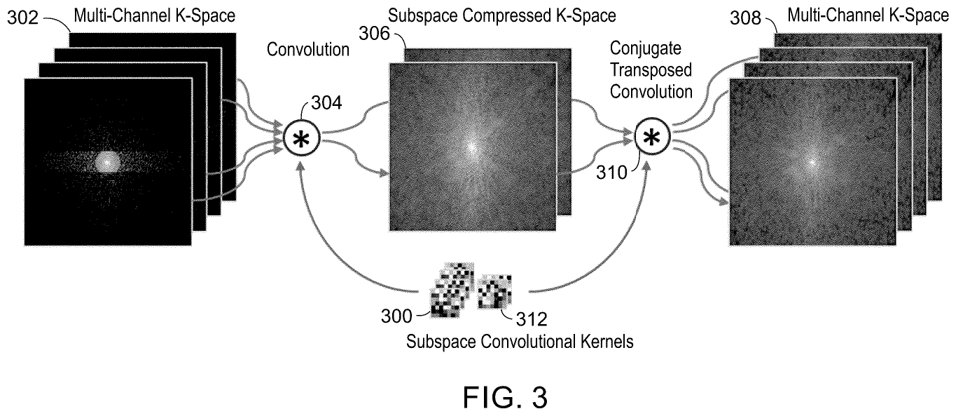

FIG. 3 is a schematic diagram of an overview of parallel imaging in k-space. The approach utilizes convolutional kernels 300 (e.g., subspace convolutional kernels or to combine information from neighboring k-space locations and from multiple channels (e.g., of physical coils or channel compressed coils). In particular, full multi-channel k-space data 302 is convolved (e.g., mapped down) with the convolutional kernels 300 (as indicated by reference numeral 304) to mix the information in the local neighborhood and across the channels to generate subspace compressed k-space data 306. However, unlike techniques such as SPIRIT or ARC, the number of channels is reduced. This forces the data into a highly compressed space similar to but not identical channel combination. The subspace compressed k-space data 306 could be as few as one channel but is less than the number of original channels utilized in acquiring the full multi-channel k-space data 302 (i.e., the originally acquired multi-channel k-space data).

As depicted, full multi-channel k-space data 308 (e.g., restored multi-channel k-space down) can be restored via deconvolution (e.g., conjugate transposed convolution) as indicated by reference numeral 310. In particular, the same convolutional kernels 300 are complex conjugated. The complex conjugates 312 of the convolution kernels 300 are utilized in the transposed convolution. New kernels may also be learned as described in greater detail below. The full cycle from the multi-channel k-space data 302, to the subspace compressed k-space data 306, and back to the multi-channel k-space data 308 represents simultaneous consistency with parallel imaging and data compressibility.

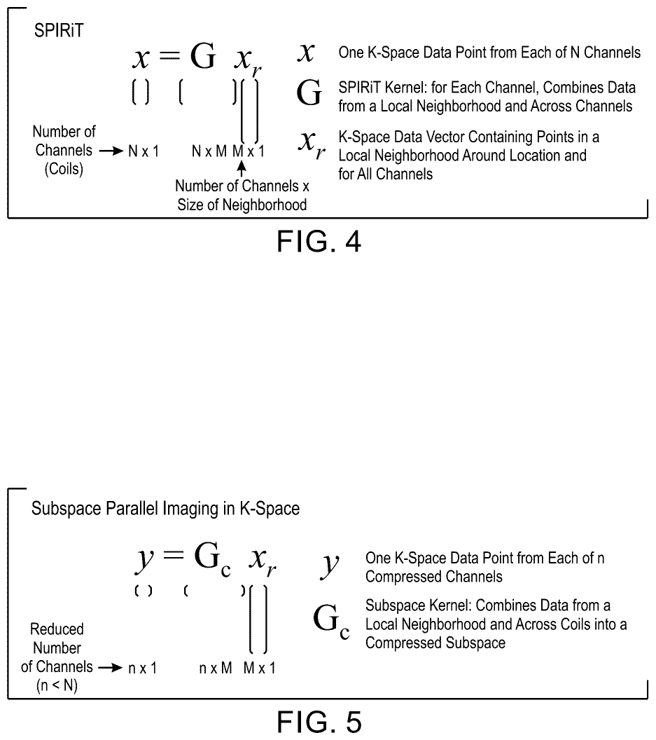

FIGS. 4 and 5 illustrate a comparison of SPIRIT and subspace parallel imaging in k-space as described in the present disclosure. FIG. 4 is a schematic diagram of SPIRIT. In SPIRIT, a matrix G performs a linear combination of k-space values in a local neighborhood and across channels (xx) to estimate a data point in each channel (x) as depicted in following equation (also depicted in FIG. 4):

$$x = G {x}_{r} . \qquad ( 1 )$$

In practice, it is implemented as a convolution, where G is a series of kernels and x is the k-space array as shown in the following equation:

$$x = G x . \qquad ( 2 )$$

The application of the operation G on x is the same as synthesizing every point from its neighborhood. In particular, if x is the correct solution, then synthesizing every point from its neighborhood should yield exactly the same k-space data.

FIG. 5 is a schematic diagram of subspace parallel imaging in k-space. In the following approach, a modified kernel compresses the result to a smaller number of channels as depicted in the following equation (also depicted in FIG. 5):

$$y = {G}_{c} {x}_{r} , \qquad ( 3 )$$

where y is one k-space data point from each of n compressed coils (where n is the number of compressed coils). In practice, the approach is implemented as a convolution, where G_{c} is a series of kernels and x is the k-space array as depicted in the following equation:

$$y = {G}_{c} x . \qquad ( 4 )$$

The full multi-channel k-space can be restored via deconvolution with the same kernel weights (denoted by

$${G}_{c}^{*} )$$

with

$$x = {G}_{c}^{*} y \qquad ( 5 )$$

leading to:

$$x = {G}_{c}^{*} {G}_{c} x . \qquad ( 6 )$$

This provides an analogous formulation to SPIRIT but now the kernels are decomposed into low rank-sub kernels that map to and from a subspace.

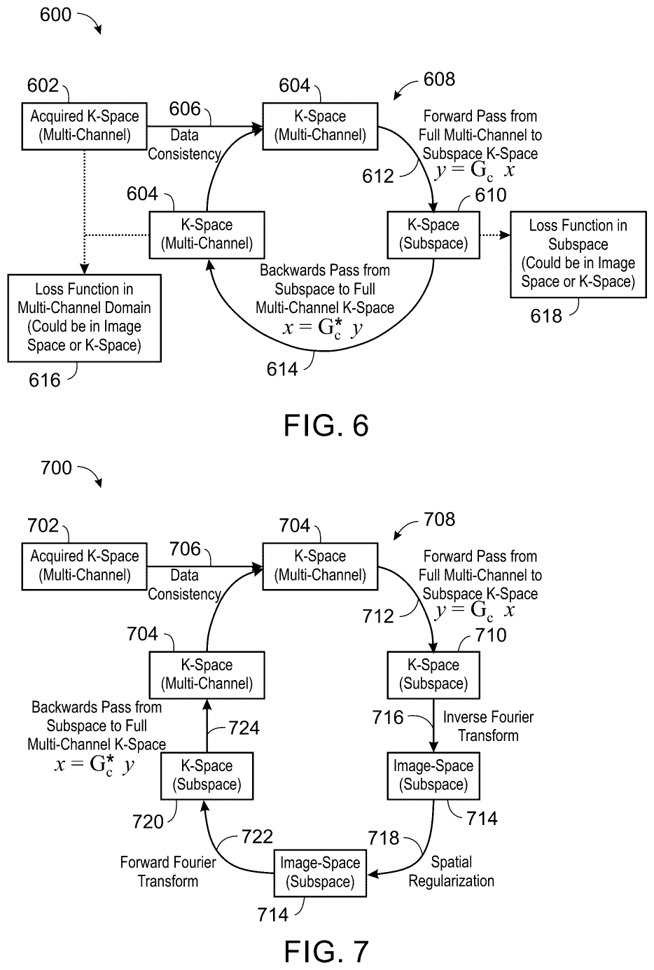

FIG. 6 is a schematic diagram illustrating a process or method 600 for learning or obtaining kernels (e.g., subspace convolutional kernels). The kernels are specific to each dataset/slice and must be adapted. The process 600 depicted in FIG. 6 illustrates obtaining kernel weights and reconstructing undersampled k-space. In particular, in the process 600, the kernels are treated as trainable parameters and are learnt in an iterative reconstruction (e.g., projection onto convex sets (POCS) iterative reconstruction).

Full multi-channel k-space data 602 (e.g., originally acquired k-space data) is acquired of a subject (e.g., patient) with an MR scanner. The multi-channel k-space data 602 may be manipulated to get rid of some data to provide undersampled multi-channel k-space data (e.g., to provide loss). The multi-channel k-space data 602 in the process 600 is undersampled. In the first cycle, multi-channel k-space data 604 is the multi-channel-space data 602. In subsequent cycles, multi-channel k-space data 604 is the reconstructed (e.g., restored k-space data). In certain embodiments, the multi-channel data 604 has had loss function applied. The multi-channel k-space data 602 provides data consistency as indicated by reference numeral 606 with reconstructed multi-channel k-space data 604. The process 600 enables obtaining kernel weights that provide consistency when the multi-channel k-space data is mapped down to the compressed subspace and back to the full multi-channel space.

Each cycle 608 includes forward passing (e.g., mapping down) the full multi-channel k-space data 604 to subspace compressed k-space data 610 utilizing equation 4 as indicated by reference numeral 612. Each cycle 608 also includes backward passing from the subspace compressed k-space data 610 to the full multi k-space data 604 (e.g., expanded utilizing equation 5) as indicated by reference numeral 614. Each cycle 608 further includes a data consistency step 606.

After certain number of cycles of data consistency, forward passes, and backward passes, loss functions may be applied in one or more locations. The number of cycles may be fixed number of iterations or determined by when the change is small enough. As depicted, a loss function may be applied between the acquired k-space data 602 and the reconstructed k-space data 604 as indicated by reference numeral 616 (e.g., prior to the data consistency step 606). In particular, loss function is applied in the multi-channel domain (e.g., in the image space or the k-space). This ensures data is unchanged following a forward and backward pass.

A loss function may also be applied on the compressed subspace data (e.g., in the image space or the k-space) as indicated by reference numeral 618. This subspace is not uniquely defined so there is flexibility to promote desirable features via application of the loss function. For example, smooth image phase or minimum entropy of gradients in the image space may be utilized.

A loss function may also be applied on the kernels themselves. Norm constraints (e.g., 11 or 12) and/or orthogonality constraints can be applied to the kernels. Kernel parameters (e.g., weights) are updated via gradient back projection (e.g., utilizing an optimizer like adaptive moment estimation (ADAM)).

FIG. 7 is a schematic diagram illustrating a process or method 700 for utilizing kernels (e.g., subspace convolutional kernels). Once the kernels are known (e.g., utilizing the process 600 in FIG. 6), they are well suited for used in various types of reconstruction (e.g., iterative reconstruction, unrolled model-based reconstruction, etc.). Compact kernels enable efficient convolutional implementations. A small subspace (e.g., typically 1 to 4 channels versus 16 plus channels) requires a smaller number of Fourier transforms to convert from compressed k-space to compressed image space. The compressed image space is well suited to regularization. For example, generic algorithms such as total variation or wavelet may be utilized for regularization. In another example, artificial intelligence-based methods utilizing convolutional neural networks may be utilized for regularization.

The process 700, utilizing iterative POCS reconstruction is one way to utilize the kernels. The kernels, G_{c}, are assumed to be known (e.g., obtained utilizing the equations 3 and 4). The subspace kernels map k-space to a small number of channels which are converted to a small number of images that undergo regularization and are then converted back to k-space. The transposed convolution maps the subspace k-space back to full multi-channel k-space, which undergoes a data consistency operation. After several iterations, the algorithm returns the full multi-channel k-space, either before or after data consistency.

Full multi-channel k-space data 702 (e.g., originally acquired k-space data) is acquired of a subject (e.g., patient) with an MR scanner. The multi-channel k-space data 702 may be manipulated to get rid of some data to provide undersampled multi-channel k-space data (e.g., to provide loss). The multi-channel k-space data 702 in the process 700 is undersampled. In the first cycle, multi-channel k-space data 704 is the multi-channel k-space data 702. In subsequent cycles, multi-channel k-space data 704 is the reconstructed (e.g., restored k-space data). The multi-channel k-space data 702 provides data consistency as indicated by reference numeral 706 with reconstructed multi-channel k-space data 704.

Each cycle or iteration 708 includes forward passing (e.g., mapping down) the full multi-channel k-space data 704 to subspace compressed k-space data 710 utilizing equation 4 as indicated by reference numeral 712. Each cycle 708 also includes performing inverse Fourier transformation on the subspace compressed k-space data 710 to generate subspace compressed image data or images 714 as indicated by reference numeral 716. The performance of inverse Fourier transformation utilizes a smaller number of inverse Fourier transforms than a number of inverse Fourier transforms needed to generate image data from the multi-channel k-space data 702 as originally acquired. In certain embodiments, spatial regularization is performed on the subspace compressed image data 714 as indicated by reference numeral 718. In certain embodiments, spatial regularization is not performed on the subspace compressed image data.

Each cycle 708 further includes performing Fast Fourier transformation on the subspace compressed image data 714 (e.g., spatially regularized or not) to generate subspace compressed k-space data 720 as indicated by reference numeral 722. Each cycle 708 further includes backward passing from the subspace compressed k-space data 720 to the full multi k-space data 704 (e.g., expanded back utilizing equation 5) as indicated by reference numeral 724. Each cycle 708 further includes a data consistency step 706.

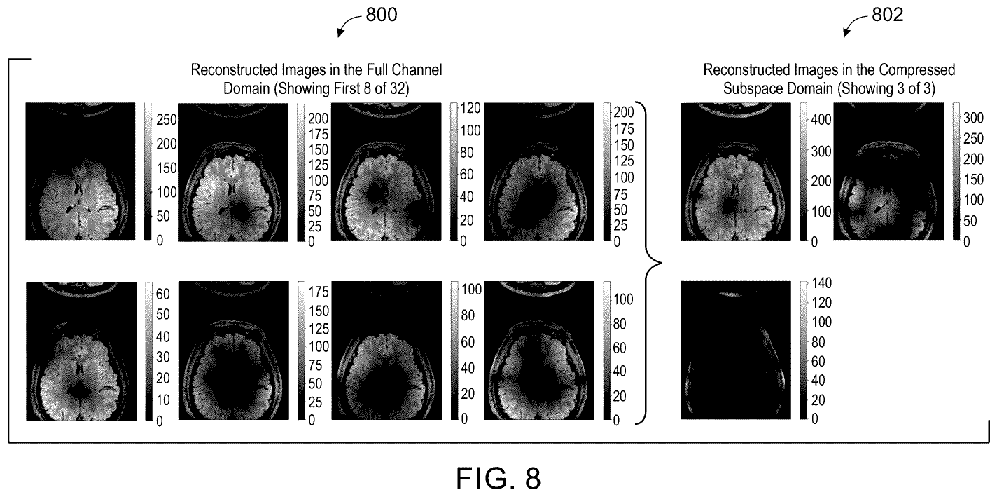

FIG. 8 depicts reconstructed multi-channel images. A left side 800 of FIG. 8 depicts reconstructed images in the full channel domain. In particular, the images on the left side 800 are from the first 8 channels of 32 channels. Volumetric channel compression was applied in generating the images on the left side 800. A right side 802 of FIG. 8 depicts reconstructed images (from the same data) in the compressed subspace data (e.g., subspace compressed image data or images 714). The images on the right side 802 are from a rank 3 reconstruction. A substantial concentration of signal is achieved in the images on the right side 802. The subspace is similar to channel combination although performed in k-space. The subspace typically requires more than a single image (i.e., rank>1) to capture relevant signals due to possible aliasing and finite kernel size.

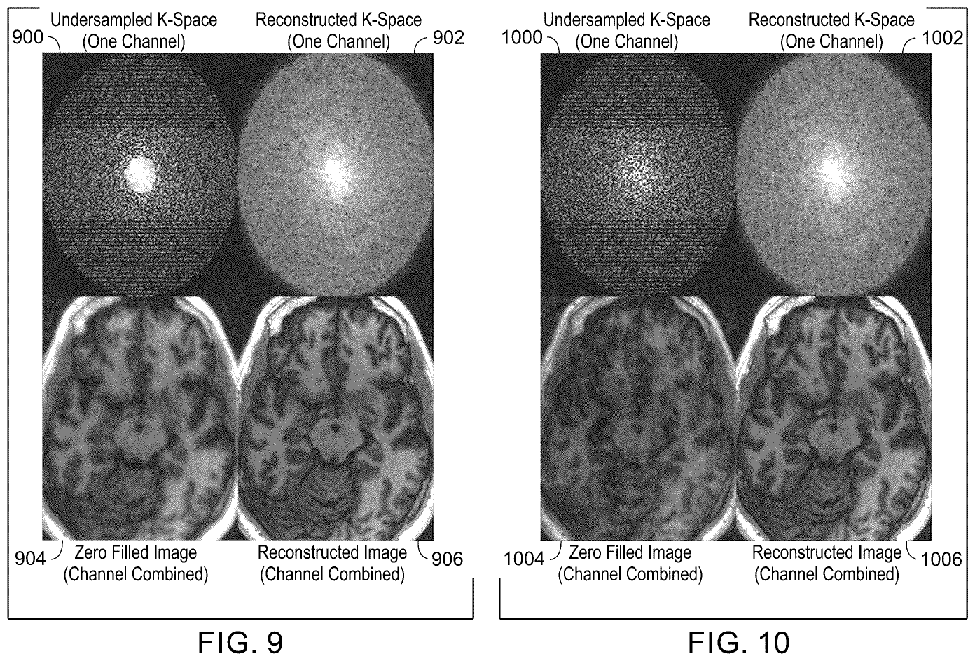

FIG. 9 depicts sample k-space and image space before and after reconstruction with subspace parallel imaging (e.g., with fully sampled calibration region) as disclosed herein derived from the same data. Image 900 is of undersampled k-space for one channel. Image 902 is reconstructed k-space for the one channel. Image 904 is a zero-filled image that is channel combined. Image 906 is a reconstructed image that is channel combined. Missing k-space locations are estimated through parallel imaging and the resulting images and have minimal aliasing.

FIG. 10 depicts sample k-space and image space before and after reconstruction with subspace parallel imaging (e.g., with undersampled calibration region) as disclosed herein derived from the same data. Image 1000 is of undersampled k-space for one channel. Image 1002 is reconstructed k-space for the one channel. Image 1004 is a zero-filled image that is channel combined. Image 1006 is a reconstructed image that is channel combined. Missing k-space locations are estimated through parallel imaging and the resulting images and have minimal aliasing. As depicted in FIG. 10, a fully sampled calibration region is not needed with the calibration method disclosed herein.

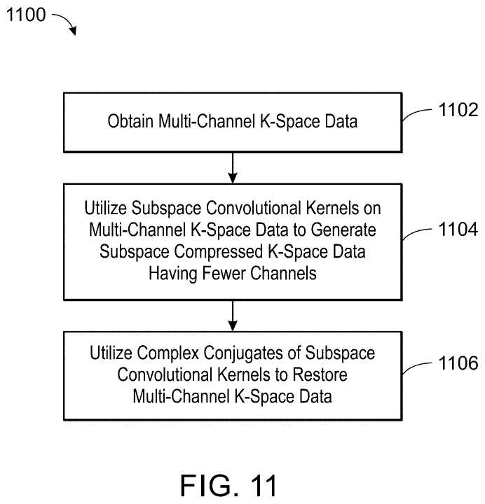

FIG. 11 illustrates a flow diagram of a method 1100 for subspace parallel imaging. One or more steps of the method 1100 may be performed by processing circuitry of the magnetic resonance imaging system 100 in FIG. 1 or a remote computing device.

The method 1100 includes obtaining multi-channel k-space data of a subject acquired with a magnetic resonance imaging scanner (block 1102). The method 1100 also includes utilizing subspace convolutional kernels on the multi-channel k-space data to combine information from local neighboring k-space locations and across multiple channels to generate subspace compressed k-space data having fewer channels than a number of channels utilized to acquire the multi-channel k-space data (block 1104). The method 1100 further includes utilizing complex conjugates of the subspace convolutional kernels to perform transposed convolution on the subspace compressed k-space data to restore the multi-channel k-space data (block 1106).

Technical effects of the disclosed subject matter include combining the benefit of k-space methods (e.g., robust and dispersed artifacts) with the benefit of image space methods (e.g., constraining the solution to a subspace). Technical effects of the disclosed subject matter include providing considerable benefit for iterative model-based methods compared to existing methods. Technical effects of the disclosed subject matter include allowing for reconstruction algorithms that produce better quality images that are robust to aliasing issues. Technical effects of the disclosed subject matter include allowing for higher net acceleration rates than are currently possible. Technical effects of the disclosed subject matter include enabling faster reconstruction speed since convolutions with compact kernels can be quite fast. In addition, there is no memory overhead of storing large sensitively maps. Further, Fourier transforms are only done in the subspace rather than the full (i.e., all of the channels) multi-channel space, thus, enabling a savings of ten times or more in the Fourier transform time. Technical effects of the disclosed subject matter include, for the patient, providing faster scans, obtaining better images, and providing more appropriate protocols covering only the anatomy of interest.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform] ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method, comprising:
obtaining, via a processing system comprising one or more processors, multi-channel k-space data of a subject acquired with a magnetic resonance imaging scanner; and
utilizing, via the processing system, subspace convolutional kernels on the multi-channel k-space data to combine information from local neighboring k-space locations and across multiple channels to generate subspace compressed k-space data having fewer channels than a number of channels utilized to acquire the multi-channel k-space data.

2. The computer-implemented method of claim 1, further comprising utilizing, via the processing system, complex conjugates of the subspace convolutional kernels to perform transposed convolution on the subspace compressed k-space data to restore the multi-channel k-space data.

3. The computer-implemented method of claim 2, wherein the subspace convolutional kernels comprise weights that provide consistency when the multi-channel k-space data is mapped down to the subspace compressed k-space data and the subspace compressed k-space data is restored to the multi-channel k-space data.

4. The computer-implemented method of claim 3, wherein the multi-channel k-space data as originally acquired provides data consistency with the multi-channel k-space data after restoration.

5. The computer-implemented method of claim 4, wherein the subspace convolution kernels are learnable.

6. The computer-implemented method of claim 5, wherein the multi-channel k-space data is undersampled.

7. The computer-implemented method of claim 6, further comprising:
in an iterative manner for a certain number of cycles:
performing, via the processing system, data consistency on the multi-channel k-space data after restoration;
mapping down, via the processing system, the multi-channel k-space data after restoration to the subspace compressed k-space data utilizing the subspace convolutional kernels; and
restoring, via the processing system, the subspace compressed k-space data to the multi-channel k-space data utilizing the complex conjugates of the subspace convolutional kernels; and
upon reaching the certain number of cycles, applying, via the processing system, a loss function in one or more locations.

8. The computer-implemented method of claim 7, further comprising applying the loss function in a multi-channel domain between the multi-channel k-space data as originally acquired and the multi-channel k-space data after restoration.

9. The computer-implemented method of claim 7, further comprising applying the loss function on subspace compressed data.

10. The computer-implemented method of claim 7, further comprising applying the loss function on the subspace convolutional kernels.

11. The computer-implemented method of claim 7, further comprising updating, via the processing system, parameters or weights of the subspace convolutional kernels utilizing gradient back projection upon reaching the certain number of cycles.

12. The computer-implemented method of claim 2, further comprising performing, via the processing system, inverse Fourier transformation on the subspace compressed k-space data to generate subspace compressed image data utilizing a smaller number of inverse Fourier transforms than a number of inverse Fourier transforms needed to generate image data from the multi-channel k-space data as originally acquired.

13. The computer-implemented method of claim 12, wherein the performing inverse Fourier transformation is part of iterative reconstruction or model-based reconstruction.

14. The computer-implemented method of claim 12, further comprising performing, via the processing system, spatial regularization on the subspace compressed image data.

15. The computer-implemented method of claim 14, further comprising performing, via the processing system, forward Fourier transformation on the subspace compressed image data after spatial regularization to generate the subspace compressed k-space data.

16. The computer-implemented method of claim 12, further comprising performing, via the processing system, forward Fourier transformation on the subspace compressed image data to generate the subspace compressed k-space data.

17. A system, comprising:
a memory encoding processor-executable routines; and
a processing system comprising one or more processors and configured to access the memory and to execute the processor-executable routines, wherein the processor-executable routines, when executed by the processing system, cause the processing system to:
obtain multi-channel k-space data of a subject acquired with a magnetic resonance imaging scanner; and
utilize subspace convolutional kernels on the multi-channel k-space data to combine information from local neighboring k-space locations and across multiple channels to generate subspace compressed k-space data having fewer channels than a number of channels utilized to acquire the multi-channel k-space data.

18. The system of claim 17, wherein the processor-executable routines, when executed by the processing system, further cause the processing system to utilize complex conjugates of the subspace convolutional kernels to perform transposed convolution on the subspace compressed k-space data to restore the multi-channel k-space data.

19. A non-transitory computer-readable medium, the non-transitory computer-readable medium comprising processor-executable code that when executed by a processing system comprising one or more processors, causes the processing system to:
obtain multi-channel k-space data of a subject acquired with a magnetic resonance imaging scanner; and
utilize subspace convolutional kernels on the multi-channel k-space data to combine information from local neighboring k-space locations and across multiple channels to generate subspace compressed k-space data having fewer channels than a number of channels utilized to acquire the multi-channel k-space data.

20. The non-transitory computer-readable medium of claim 19, wherein the processor-executable code, when executed by the processing system, further causes the processing system to utilize complex conjugates of the subspace convolutional kernels to perform transposed convolution on the subspace compressed k-space data to restore the multi-channel k-space data.
